(19) United States
(12) Patent Application Publication
(10) Pub. No.: US 2003/0195262 A1
(43) Pub. Date: Oct. 16, 2003

(54) Use of acetone as an inhibiting, or neutralizing, agent of diseased cells

(76) Inventor: Patricia Marlene Williams, Overland Park, KS (US)

Correspondence Address: Patricia M. Williams, 11732 West 108th Circle, Overland Park, KS, 66210-1234, US

(21) Appl. No.: 10/408,859

(22) Filed: Apr. 8, 2003

Related U.S. Application Data
(60) Provisional application No. 60/373,066, filed on Apr. 16, 2002.

(51) Int. Cl. A61K 31/12

(52) U.S. Cl. 514/675

(57) ABSTRACT

This invention relates to the use of acetone as an agent to selectively destruct, neutralize, and/or inhibit cells infected by viral infections, solid tumor cancers, and carcinomas. The treatment method allows the acetone to be presented to the diseased cells in a direct manner, thereby causing the least amount of disturbance to the otherwise healthy body.

### 1. FIELD OF THE INVENTION

This invention relates to a method of selectively destroying, or inhibiting a cell or cells infected by a virus or cancer.

### 2. BACKGROUND OF THE INVENTION

In recent years many forms of treatment have been proposed for viral diseases and cancer. Due to the many differences that separate these pathogens, including, their structure, their method of replication and their susceptibility to or resistance to various treatment modalities, a single method of inhibiting viral infections and solid tumor cancers would not be considered feasible. However, due to the efficacy of a known compound in affecting the lipid surround of the virus or cancer cell and the diseased cell, a realistic cure for these diseases has become evident.

Acetone, an organic solvent, has many uses in medical and non-medical fields. In most cases it is used as a drying or cleaning agent, or to facilitate the breaking down of other substances. When used with some precaution, it is handled every day by professionals and non-professionals alike. Small amounts used on the human body, as in the case of nail polish remover have not provided concern about its use.

### 3. SUMMARY OF THE INVENTION

It is the object of this invention to provide an effective method of treatment for viral infections, solid tumor cancers, and carcinomas. The preferred embodiment is to treat the diseased cells directly, thereby relieving the patient of the undue stress currently in practice of treating not only the diseased cells, but also larger surrounding areas, the entire body, and/or surgery. By means of this invention acetone would be administered as a drying agent to the lipid surround of the diseased cells effectively rending the disease vulnerable to further destructive action by the acetone and the body's own defensive actions. Acetone is extremely effective, usually requiring only one treatment, in a predetermined dose, to effectively destroy or neutralize the diseased cells.

### DESCRIPTION

The invention is the use of acetone, C3H6O, as an inhibitor, or neutralizer, of diseased cells in human and veterinary medicine. Said diseased cells to include, but not be limited to viral infections, solid tumor cancers, and carcinomas. The acetone should be used alone, or with suitable carriers in predetermined doses. Said carriers to include, but not be limited to, water, emollients, an anesthetizing agent, and/or pharmaceutically acceptable compounds to administer the acetone therapeutically. The therapeutic treatments may include topical; with or without light abrasive action, parentally, injection, or by means of a suppository, depending on whether the preparation is used to treat internal or external diseased cells.

What is claimed is:

1. Topical treatments of 100% acetone, or with pharmaceutically acceptable carriers to administer the acetone therapeutically, applied in a predetermined dosage, preferably with a light abrasive action, to the site of an active viral infection, including but not limited to, Herpes Simples I or II will inhibit or neutralize the disease.

2. Acetone, with or without pharmaceutically acceptable carriers to administer the acetone therapeutically, introduced to solid tumor cancers or carcinomas in a predetermined dosage, either into the lipid cell surround and/or the cancer cells will inhibit or neutralize the diseased cells.
